(19) United States
(12) Patent Application Publication
(10) Pub. No.: US 2017/0073723 A1
(43) Pub. Date: Mar. 16, 2017

(54) METHOD OF DETECTING MICROBES CARRIED IN A NON-LIQUID SAMPLE

(71) Applicant: MERCK PATENT GMBH, Darmstadt (DE)

(72) Inventors: Wolfgang SCHMIDT, Weiterstadt (DE); Tim CSER, Roseville, CA (US)

(73) Assignee: MERCK PATENT GMBH, Darmstadt (DE)

(21) Appl. No.: 15/121,944

(22) PCT Filed: Feb. 25, 2015
(86) PCT No.: PCT/EP2015/000434

§ 371 Date: Aug. 26, 2016

Related U.S. Application Data
(60) Provisional application No. 61/944,853, filed on Feb. 26, 2014.

(51) Int. Cl. C12Q 1/06; C12Q 1/14

(52) U.S. Cl. CPC C12Q 1/06; C12Q 1/14; G01N 2333/195

(57) ABSTRACT

A method and system of preparing a sample for microbial testing by providing a sample in a sample bag, a dry sterile growth medium in a bag, and a source of pure (Type II) water heated to a temperature of about 25-50° C., homogenizing the sample, reconstituting the dry medium with the heated Type II water, mixing the medium and sample to a homogenous consistency and incubating the sample for a period of time sufficient to allow the growth of microbes. A tankless water heater or a heat exchanger can be used to heat the water to a desired temperature. The Type II water can be heated either before or after it is made. A blender can be used for the sample homogenization as well as the final mixing before incubation and may be a paddle or stationary blender. Microbes are detected by various methods well known in the art.

The present invention relates to a method of detecting microbes in a non-liquid (solid or semisolid) sample such as food (vegetables, meats, dairy, etc), environmental samples (sponges, swabs, soil, etc) or cosmetic or pharmaceutical preparation (creams, ointments, etc) and various systems for doing so. More particularly it relates to a method of detecting, enumerating and optionally identifying microbes in a food sample using an irradiated dehydrated growth medium which is reconstituted by heated pure, sterile water and systems for doing so.

### BACKGROUND

Product safety is an issue of concern and tests are routinely done on food and cosmetic products to determine the presence or absence of microbes such as bacteria, molds, and yeasts that may affect the health of the entity (human, animal) or adversely affect the shelf life of the food product. The products such as food can be in fresh form, canned, bottled, frozen, dried and other typical forms in which food is generally provided. While the discussion in this application shall reference food it should be clear that the present invention is also applicable to other non-liquid samples such as cosmetics and pharmaceutical preparations and soil and other environmental samples.

A typical process for determining whether the food product is safe is to take a sample of the food and homogenize it in liquid culture medium using a stationary blender such as a Waring® blender or more preferably in a paddle blender such as a Stomacher® blender (available from Seward Limited) so that as many microbes as possible are driven into suspension and thus able to grow in the presence of a suitable growth medium.

The medium is generally provided in bulk liquid or dehydrated form, such as a powder or granules. The dehydrated medium needs to be reconstituted with water before use. Both forms are then autoclaved to ensure that the medium is sterile before use so that one minimizes the potential for a false positive in the test with the food sample.

The autoclaved medium is allowed to cool before the sample of food is mixed with it to minimize any damage that a superheated medium might do to the microbes in the sample. The food sample and medium are then mixed until homogenous and then placed into an incubator to come to a suitable temperature for microbial growth and for a sufficient time to allow that growth to occur so that any microbes are present in the sample can be detected.

Various methods are well known for detecting the presence or absence and even the identity of microbes in a sample.

There are several disadvantages to the present method and systems. Bulk medium needs to have a desired amount withdrawn for use. For dehydrated media, that desired amount needs to be reconstituted with water. The bulk medium, either liquid or reconstituted dehydrated medium, then needs to be sterilized using an autoclave.

The use of the autoclave step is time consuming, both in its duration to ensure sterility as well as in its cool down time so that the autoclaved medium can be used without adversely affecting the microbes that may be present in the sample.

Anecdotally, the autoclave step can adversely affect certain components of the medium which are heat liable making the medium less efficient and potentially requiring additional incubation time or allowing for false negative test results.

The time of incubation is also time consuming as it involves first raising the sample and medium to the proper incubation temperature (typically 25° to 50° C.) and then allowing the microbes to grow at their selected optimum temperature. The heating step, depending on a number of factors such as the size of the sample and the ability of the incubator to provide heat into that sample, can add several hours to the process.

Moreover, the food or other material from which the sample has been taken is generally kept isolated until the test has been completed and it is deemed safe. This keeps food, cosmetics or pharmaceutical preparations off the market and adds to the cost of storage for that food while awaiting the results.

What is needed is a better method and system for preparing such food samples for testing.

### SUMMARY OF THE INVENTION

The present invention relates to a method and a system for the method of preparing samples for microbial testing. The method uses a sterile medium, such as an irradiated dehydrated medium, in particular a gamma irradiated dehydrated medium (in powder granulated format with or without included selective supplements), in a sealed sterile pouch, in a predosed amount for different sample sizes from 1 g to 500 g. Heated pure, sterile water (Type II water) in the range of 25°-50° C., preferably 30°-45° C., more preferably 37°-42° C. is provided and added to the dehydrated medium to reconstitute it and the reconstituted medium and food sample are homogenized either during the reconstitution of the medium or after the reconstitution of the medium and placed into the incubator for a selected period of time to allow any microbes of interest to grow to a size suitable for detection.

In one embodiment, the method of preparing a sample for microbial testing is taught by providing a sample of food to be tested in a sample container, generally in the form of a plastic bag or a large Petri Dish-like covered plate, providing an irradiated, sterile, dehydrated growth medium, providing a source of pure (Type II) water heated to a temperature of about 25° C. to 50° C., homogenizing the sample, adding the heated water to the medium to reconstitute the medium into a liquid form either mixing the heated Type II water with the medium alone and then adding the reconstituted liquid medium to the food sample in the sample container or preferably by adding the dry medium, heated Type II water and sample together and reconstituting the medium with the sample and water and then mixing the reconstituted liquid medium and sample to a homogenous consistency and incubating the reconstituted medium and sample for a period of time sufficient to allow the growth of any microbes in the sample. A water system such as an Elix® water system or a double distillation column can be used to provide the sterile, pure (Type II) water. A tankless water heater or a heat exchanger can be used to heat the water to a desired temperature preferably between about 25° and 50° C. The Type II water can be heated after it is made or before it is made into Type II water by providing heated water to the device which creates the Type II water. A blender can be used for the sample homogenization as well as the final homogenization before incubation and may be a paddle or stationary blender. Hand kneading can replace either or both blender steps if needed.

In a further embodiment, the method of preparing a sample for microbial testing is taught by providing a sample to be tested in a sample container, providing an irradiated, sterile, dehydrated growth medium, providing a source of pure, sterile (Type II) water heated to a temperature of about 25° C. to about 50° C., homogenizing the sample, adding it to its container, adding the heated water and dehydrated medium to the food sample in the sample bag and mixing the heated water, medium and food to a homogenous consistency and incubating the bag containing the medium and food for a period of time sufficient to allow the growth of any microbes in the food sample. A water system such as an Elix® water system or a double distillation column can be used to provide the pure (Type II) water. A tankless water heater or a heat exchanger can be used to heat the water to a desired temperature preferably between about 25° and 50° C. A blender can be used for the sample homogenization as well as the final homogenization before incubation and may be a paddle or stationary blender.

In another embodiment, the system for preparing a sample for microbial testing is taught by providing an amount of irradiated, sterile, dehydrated growth medium, a system for producing pure (Type II) water, a system for heating the water, a blender for homogenizing a sample and an incubator for growing any microbes contained within the sample. A system such as an Elix® water system or a double distillation column can be use to provide the pure (Type II) water. A tankless water heater or a heat exchanger can be used to heat the water to a desired temperature preferably between about 25° and 50° C. The tankless water heater or heat exchanger can be upstream or downstream of the device that makes the Type II water. A blender can be used for the sample homogenization as well as the final homogenization before incubation and may be a paddle or stationary blender.

In one embodiment the medium is reconstituted before it is added in liquid form to the homogenized sample. Required medium aliquots can then be added to the food sample e.g. by using a dispenser and the like. Consequently, the two components are then remixed to provide a homogeneous sample before incubation.

In another embodiment, the homogenized sample, heated pure water and dehydrated medium are mixed together to allow the medium to reconstitute itself into a liquid and to mix the components for incubation.

In the Figures

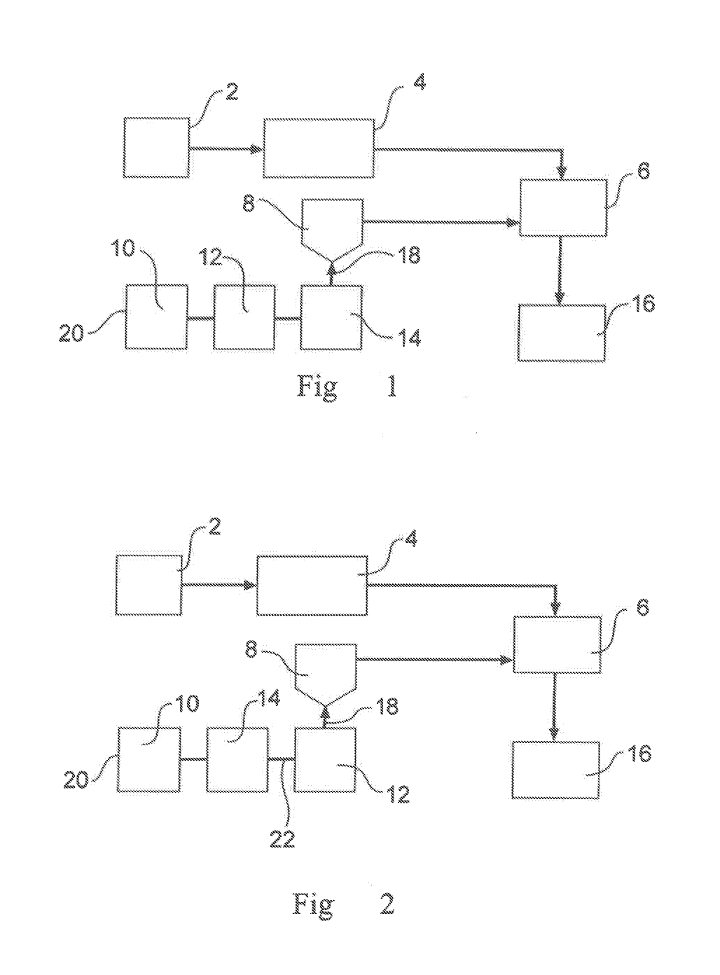

FIG. 1 shows a system and method according to a first embodiment of the present invention.

FIG. 2 shows a system and method according to a second embodiment of the present invention.

FIG. 3 shows a system and method according to a third embodiment of the present invention.

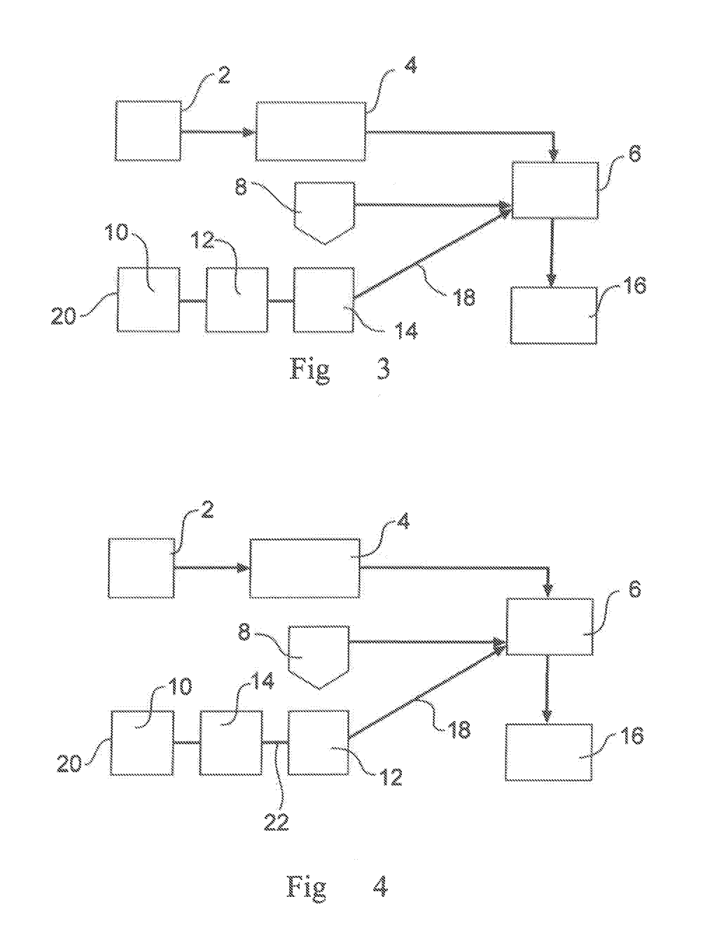

FIG. 4 shows a system and method according to a fourth embodiment of the present invention.

### DETAILED DESCRIPTION

FIG. 1 shows a first embodiment of the system and method of the present invention. A sample 2 is homogenized 4 to create a homogenous sample mass. In many cases, the mass is at least partially liquefied as well by the liquid that is contained within the sample itself.

Separately, a sterile dry medium 8, such as a sterile irradiated dehydrated medium is obtained. This may be from a bulk supply and placed into a closed container or more preferably is supplied in a closed premeasured bag such as the ReadyBag™ medium available from Merck KGaA.

Additionally, heated Type II water 18 is prepared for use in reconstituting the medium 8.

In this embodiment, untreated water 10 is supplied from a source 20 to a heating device 12 such as a tankless water heater or a heat exchanger which brings the untreated water 10 to a desired temperature, typically between about 25° C. and 50° C., preferably between 30°-40° C. and more preferably between 32° and 37° C.

The heated water 10 is then treated in a device 14 for making Type II water to form the heated Type II water 18.

This heated Type II water 18 is then added to the medium 8 and the medium is reconstituted.

In some instances, a mixing step may be optionally required to ensure that the medium 8 is completely dissolved.

The amount of heated Type II water 18 is dependent on the medium 8 supplied, its amount and the required volume of water for that amount of medium 8 needed to dissolve the medium 8. This is generally information provided by the medium supplier. In those cases where it is not provided, a simple test can determine the amount of water needed per gram of dry medium 8 and this can then be extrapolated to the amount needed for the test.

It is preferred that only the amount of Type II water needed to reconstitute the medium 8 be heated and used immediately.

Alternatively, heated Type II water 18 may be placed in an insulated storage container such as thermal flask or a jacketed reservoir for later use. The jacket is at least insulative in nature and in some cases may be heated so as to help reduce any heat loss during storage.

The reconstituted heated medium 8/18 is then mixed with the homogenized sample 4 to create a blended sample/medium material 6. This material 6 can be placed into a sample bag or container and is then incubated in an incubator 16 for a period of time to determine whether any microbes may be present. The incubator 16 is generally kept at an elevated temperature between about 25° C. and 50° C. to help foster the growth of any microbes that are present in the sample 6. The sample/medium blend 6 after incubate for a suitable period of time can then be tested to determine the presence or absence of microbes as described below.

FIG. 2 shows a second embodiment of the present invention. To the extent that features in this Figure are the same as those of FIG. 1, the same reference numerals are used to delineate them.

In this embodiment, the Type II water 18 is heated after its formation. Again such a step can be accomplished in a heating device 12 such as a tankless water heater or a heat exchanger which brings the Type II water 22 to a temperature between about 25° C. and 50° C. and the Type II heated water 18 is then supplied to the medium bag 8 for reconstitution of the dry medium 8.

It is preferred that only the amount of water needed to reconstitute the medium 8 be heated and used immediately. Alternatively, the heated Type II water 18 may be placed in an insulated storage container such as thermal flask or a jacketed reservoir for later use. The jacket is at least insulative in nature and in some cases may be heated so as to help reduce any heat loss during storage.

The reconstituted heated medium 8/18 is then mixed with the homogenized sample 4 to create a blended sample/medium material 6. This material 6 can be placed into a sample bag or container and is then incubated in an incubator 16 for a period of time to determine whether any microbes may be present. The incubator 16 is generally kept at an elevated temperature between about 25° C. and 50° C. to help foster the growth of any microbes that are present in the sample. The sample/medium blend 6 can then be tested to determine the presence or absence of microbes as described below.

FIG. 3 shows another embodiment of the present invention. To the extent that features in this Figure are the same as those of FIG. 1, the same reference numerals are used to delineate them.

In this embodiment, the medium 8 is reconstituted with the heated Type II water 18 when it is mixed with both the Type II water 18 and the homogenized sample 4 in a container such as a bag or plate 6. All steps after the mixing of the medium, water 18 and homogenized sample 4 are the same as those of FIGS. 1 and 2.

As shown the water 10 is heated in a heating device 12 before it is treated to become a Type II water 18 as in FIG. 1.

FIG. 4 shows another embodiment of the present invention. To the extent that features in this Figure are the same as those of FIG. 1, the same reference numerals are used to delineate them.

In this embodiment, the medium 8 is reconstituted with the heated Type II water 18 when it is mixed with both the heated Type II water 18 and the homogenized sample 4 in one container 6.

As shown the water 10 is heated in a device 12 after it is treated to become a Type II water 18 as in FIG. 1.

The microbes to be detected by the present systems and methods are selected from the group consisting of yeast, mold and bacteria. In particular are those microbes known as food and beverage spoilage and food pathogenic microorganisms to cause issues with humans or animals that may ingest them. These include but are not limited coliform bacteria, Escherichia coli including pathogenic E. coli such as Shigatoxin producing E. coli (STEC), Salmonella, Listeria, Staphylococci, Shigella, Vibrio and the like. In addition the invention covers culture media application for Pharmaceutical and Personal Care product related quality and release testing.

Pure water means a Type II water which typically has a resistivity of greater than 5 milliohms per centimeter at a measured temperature of 25° C. (>5 MΩcm at 25° C.). Such water is a standard used in laboratories to prepare media and conduct other such duties. It typically has low levels of organics, microorganisms and particles due to the process used to make it, making it desirable for aseptic and sterile applications. If desired, an additional sterile grade filter (0.2 micron or even a 0.1 micron nominal pore size filter such as Durapore® filters or Millipore Express® filters from EMD Millipore Corporation, Billerica, Mass.) may be used downstream of the type II water making device or the heater whichever is the latter. It can be made by a system which first filters the water by a reverse osmosis process through a reverse osmosis membrane or membranes to create a reverse osmosis (RO) water and then further treats the RO water to a deionization step and preferably a UV step as well. One such system is known as the Elix® system available from EMD Millipore Corporation of Billerica, Mass.

Alternatively, it can be made by a double distillation process where two or more distillation columns are used in series to create a Type II water.

The sample and the resultant mixture of sample, medium and water can be homogenized in a variety of ways by a variety of equipment. In its simplest form, the food is placed in a sealed bag and kneaded by hand, roller or the like to homogenize the sample and free as many microbes as possible from the confines of the food sample and into the suspension created by the homogenization. Likewise it can be crushed in a mortar and pestle arrangement.

Preferably, such homogenization is done by a blender such as a stationary blender in which the blender bowl contains a series of blades which rotate on a shaft and pulverize the sample into a paste. One such blender is a Waring-style stationary blender available from Waring Corporation. Alternatively, a paddle blender can be used. A paddle blender uses two or more paddles that rock back and forth against each other with a bag containing the sample being between them. The paddles crush the sample and drive the microbes that are present into the suspension formed by the homogenized sample. One such device is called the Stomacher® paddle blender available from Seward Limited of the UK.

The pure water needs to be heated before being added to the dehydrated medium to reconstitute it for use in the detection of microbes.

The heating system for the pure water can be upstream of the pure water manufacture such that heated water is used in the pure water manufacture process.

Alternatively, the heating can occur to already manufactured Type II water or downstream of the pure water manufacture.

This heating, either upstream or downstream of the pure water manufacture can be accomplished through the use of a tankless water heater or a heat exchanger which heats the water to a desired temperature range, typically from 25-50° C. Such systems are well known such as a tankless water heater or heat exchanger available from Keltech Incorporated or a TYTAN™ tankless water heater available from Process technology Inc of Mentor, Ohio.

Microbes, if present, are detected, for presence/absence, often enumerated and even identified.

The most basic test is to simply visually observe the incubated sample for the formation of colony forming units (CFUs) which can be detected by the human eye, alone or through a microscope. In some cases, the mixed medium sample are put onto reader plates and incubated in that manner for visual detection.

The chemical detection method typically involves the use of dyes and fluorescent materials which interact with the growing microbes during incubation and is another way for determining the presence and identity of the microbes. These can be visually detected or detected through the use of electronic inspection systems that identify the presence of the chemical entities and often their level and location as well. Such instruments can include fluorometers, microscopes, CCD and other types of video and cameras and the like.

A more recent method of detection is to use various molecular methods to determine the presence and identity of any microbes present. These systems typically amplify a selected DNA (PCR method) or RNA (TMA method) sequence with a signal moiety such as a color or a fluorophore group which are then detected visually or with the help electronic inspection equipment.

### EXAMPLES

### Example 1

A 325 gram sample of boneless chicken is selected and placed into a Waring blender. The blender is covered and the sample is processed by the blender until of a smooth consistency. The sample is decanted into a plastic reclosable style bag (ZipLock, 1 quart bag).

Separately, 4 liters of Type II water is drawn from the point of use dispenser of an Elix® pure water system (EMD Millipore Corporation) and heated in tankless water heater until it reaches a temperature of 35° C.

A bag of dehydrated irradiated buffered peptone medium containing 86 grams of dry medium is obtained (ReadyBag® Merck KGaA) and opened. 3375 ml of heated Type II water is added to the bag and the bag is resealed and lightly shook by hand to dissolve the medium. The shaking is stopped from time to time to check on the dissolution. When the medium is completely dissolved it is added to the resealable bag containing the sample. The resealable bag is placed into an incubator at 37° C. and allowed to incubate for 24 hours.

The bag is removed from the incubator and used for further detection of pathogenic bacteria or spoilage bacteria.

### Example 2

A 375 gram sample of boneless chicken is selected and placed into a Waring blender. The blender is covered and the sample is processed by the blender until of a smooth consistency. The sample is decanted into a plastic reclosable style bag (ZipLock 1 quart bag)

Separately, 4 liters of Type II water is drawn from the point of use dispenser of an Elix® pure water system (EMD Millipore Corporation) and heated in tankless water heater until it reaches a temperature of 35° C.

A bag of dehydrated irradiated buffered peptone medium containing 86 grams of dry medium is obtained (ReadyBag® Merck KGaA) and opened. 3375 ml of the heated Type II water and the medium are added to the sample bag and the bag is resealed.

The resealable bag is placed into an incubator at 37° C. and allowed to incubate for 24 hours.

The bag is removed from the incubator used for further detection of pathogenic bacteria or spoilage bacteria.

### Example 3

A 375 gram sample of boneless chicken is selected and placed into a Waring blender. The blender is covered and the sample is processed by the blender until of a smooth consistency. The sample is decanted into a plastic reclosable style bag (ZipLock 1 quart bag)

A bag of dehydrated irradiated buffered peptone medium containing 86 grams of dry medium is obtained (ReadyBag® Merck KGaA) and opened. The medium is added to the sample bag.

Separately, 4 liters of Type II water is drawn from the point of use dispenser of an Elix® pure water system (EMD Millipore Corporation) and heated in tankless water heater until it reaches a temperature of 35° C.

3375 ml of the heated Type II water are added to the sample bag comprising the sample and the medium and the bag is resealed.

The resealable bag is placed into an incubator at 37° C. and allowed to incubate for 24 hours.

The bag is removed from the incubator used for further detection of pathogenic bacteria or spoilage bacteria.

1. A method of performing a microbiological test on a semi-solid and/or solid sample comprising the steps of:
a. providing a sample to be tested,
b. providing dehydrated growth medium,
c. providing a source of pure (Type II) water heated to a temperature of about 25-50° C.
d. homogenizing the sample,
e. reconstituting the medium into a liquid form with the heated Type II water by a method selected from the group consisting of
i. adding the heated Type II water to the medium and reconstituting it and then adding the reconstituted liquid medium to the homogenized sample in a container, or
ii. adding the heated Type II water, dehydrated medium and the homogenized sample in a container together,
f. mixing the heated Type II water, medium and homogenized sample to a homogenous consistency and
g. incubating the container containing the water, medium and homogenized sample for a period of time sufficient to allow the growth of any microbes in the sample.

2. The method of claim 1 wherein the pure water is heated to a temperature between about 30-45° C., more preferably between 37 and 42° C.

3. The method of claim 1 wherein the water is heated by a heater upstream of the source of the water.

4. The method of claim 1 wherein the water is heated by a heater located downstream of the source of the water.

5. The method according to claim 1 further comprising the step (i) of examining the incubated sample to determine the presence or absence of microbes.

6. The method according to claim 1 further comprising the step (i) of examining the incubated sample to determine the presence or absence of microbes and enumerating the microbes if found.

7. The method according to claim 1 further comprising the step (j) of identifying any microbes which are present.

8. The method according to claim 1 wherein the microbes to be detected are selected from the group consisting of yeast, mold and bacteria.

9. The method according to claim 1 wherein the microbes to be detected are microbes known as food and beverage spoilage and food pathogenic microorganisms to cause issues with humans or animals that may ingest them.

10. The method according to claim 1 wherein the microbes to be detected are bacteria selected from the group consisting of coliform bacteria, Escherichia coli including pathogenic E. coli such as Shigatoxin producing E. coli (STEC), Salmonella, Listeria, Staphylococci, Shigella, Vibrio and the like.

11. The method according to claim 1 wherein the sample is incubated for a period of time to grow colony forming units that are detectable.

12. The method according to claim 1 wherein the sample is incubated for a period of time to grow colony forming units that are detectable by a detection method selected from the group consisting of visual detection, chemical detection (dyes, fluorescence), molecular detection (PCR, rPRC, TMA,) and electronic detection (laser topography, CCD camera, Camera/microscope) with or without the addition of chemical or molecular detection agents.

13. The method according to claim 1 wherein the sample is homogenized by a blender selected from the group consisting of a stationary blender and a paddle blender.

14. The method according to claim 1 wherein the dehydrated medium is in a form selected from the group consisting of powders and granules.

15. A system for detecting microbes in a sample comprising a bag of sterile granular growth medium, a system for producing pure (Type II) water, a system for heating the water, a blender for homogenizing a food sample and the combined sample, medium and water and an incubator for growing any microbes contained within the sample.

16. The method according to claim 1 wherein the water is heated to a temperature between about 25-50° C. by a heater and wherein the heater is selected from the group consisting of tankless water heaters and heat exchangers.

17. The method according to claim 1 wherein the sample is selected from the group consisting of food, environmental samples, cosmetics and pharmaceutical preparations.
